(19) United States
(12) Patent Application Publication
(10) Pub. No.: US 2008/0281386 A1
(43) Pub. Date: Nov. 13, 2008

(54) Systems and methods for treating body tissue

(75) Inventors: Mathieu Herbette, Mountain View, CA (US); Curtis Tom, San Mateo, CA (US)

Correspondence Address: PATENT OFFICE OF DR. CHUNG S. PARK, P. O. BOX 62312, SUNNYVALE, CA, 94088-2312, US

(73) Assignee: Tessaron Medical, Inc., Sunnyvale, CA (US)

(21) Appl. No.: 11/801,453

(22) Filed: May 9, 2007

(51) Int. Cl. A61B 18/04

(52) U.S. Cl. 607/103

(57) ABSTRACT

Devices for treating body tissue. The device includes: an elongated cylindrical guide portion having a distal end and a proximal end; and one or more heat generators securely attached to the elongated guide portion. The heat generators are operative to inductively generate heat energy in response to an electromagnetic field externally applied thereto. One or more of the heat generators are disposed near target tissue so that the heat energy generated by the heat generators is used to treat the target tissue during operation.

### BACKGROUND

The present disclosure generally relates to medical methods and apparatus, more particularly, to treating various types of body tissue by RF inductive heating.

Human and/or animal can suffer from various types of tissue-related illnesses, such as breast cancer and incontinence. Breast cancer may be the most common cancer that forms in tissues of the breast, usually the ducts (tubes that carry milk to the nipple) and lobules (glands that make milk). In general, breast cancer has two types: in situ and invasive. In situ breast cancer is a type of cancer in which the breast cancer cells have remained contained within their place of origin, i.e., they haven't invaded breast tissue around the duct or lobule. Invasive (infiltrating) breast cancers are those that break free of where they originate, invading the surrounding tissues that support the ducts and lobules of the breast. In some cases, the cancer cells can travel to other parts of the body, such as the lymph nodes.

Incontinence, which refers to involuntary urination, is experienced by older adults who have difficulty with bladder control usually because of either urinary tract disease, nervous system dysfunction, allergic response, ruptured disk, or psychological stress. Women tend to experience involuntary urination after childbirth, surgery, or inflammation of the urethra, while men tend to get it if they have a prostate problem.

Various types of techniques have been developed to treat abnormal tissue. For instance, one technique to treat breast cancer may be removal of the breast to provide the best assurance against recurrence of the cancer, but is disfiguring and requires the patient to make a very difficult choice and, quite often, to have a subsequent cosmetic surgery. (Hereinafter, the term cancer collectively refers to cancerous, pre-cancerous, and other abnormal cells or disease conditions.) Chemotherapy and radiation can be another technique, but cannot provide an effective assurance against recurrence. Lumpectomy can be an alternative approach, but is associated with a substantive chance of recurrent. For another instance, homeopathic treatment may be the most common approach to relieve incontinence, but does not solve the fundamental problem of the incontinence. As such, there is a strong need for a technique to provide an effective technique to treat various types of body tissue.

### SUMMARY OF THE DISCLOSURE

In one embodiment, a catheter includes: an elongated cylindrical guide portion having a distal end and a proximal end; and one or more heat generators securely attached to the elongated guide portion. The heat generators are operative to inductively generate heat energy in response to an electromagnetic field externally applied thereto. At least one of the heat generators is disposed near target tissue so that the heat energy generated by the heat generator is used to treat the target tissue during operation.

In another embodiment, a system for treating tissue includes: a coil for generating an alternating electromagnetic field; and a catheter that has an elongated cylindrical guide portion; and one or more heat generators securely attached to the elongated guide portion. The heat generators are operative to inductively generate heat energy in response to an electromagnetic field externally applied thereto. At least one of the heat generators is disposed near target tissue so that the heat energy generated by the heat generator is used to treat the target tissue during operation.

In yet another embodiment, a method for treating tissue includes the steps of: positioning a heat generator of a catheter near tissue to be treated; and applying an external electromagnetic field to the heat generator to cause said heat generator to inductively generate heat energy in response to the electromagnetic field thereby treating the tissue by the heat energy

### BRIEF DESCRIPTION OF THE DRAWINGS

FIG. 1 shows a schematic diagram of a treatment system for treating human breast cancer in accordance with one embodiment of the present invention;

FIG. 2 shows a schematic perspective view of a catheter in FIG. 1;

FIG. 3 shows a schematic cross sectional diagram of the catheter in FIG. 2, taken along the line III-III;

FIG. 4 shows a schematic cross sectional diagram of another embodiment of the catheter in FIG. 2;

FIGS. 5A-5B show schematic cross sectional diagrams of various embodiments of the tip portion of the catheter in FIG. 2;

FIGS. 5C-8 show schematic diagrams of various embodiments of the catheter in FIG. 2; and

FIG. 9 shows a schematic top view of an electrical coil in accordance with another embodiment of the present invention.

### DETAILED DESCRIPTION OF THE PREFERRED EMBODIMENTS

The following detailed description is of the best currently contemplated modes of carrying out the invention. The description is not to be taken in a limiting sense, but is made merely for the purpose of illustrating the general principles of the invention because the scope of the invention is best defined by the appended claims.

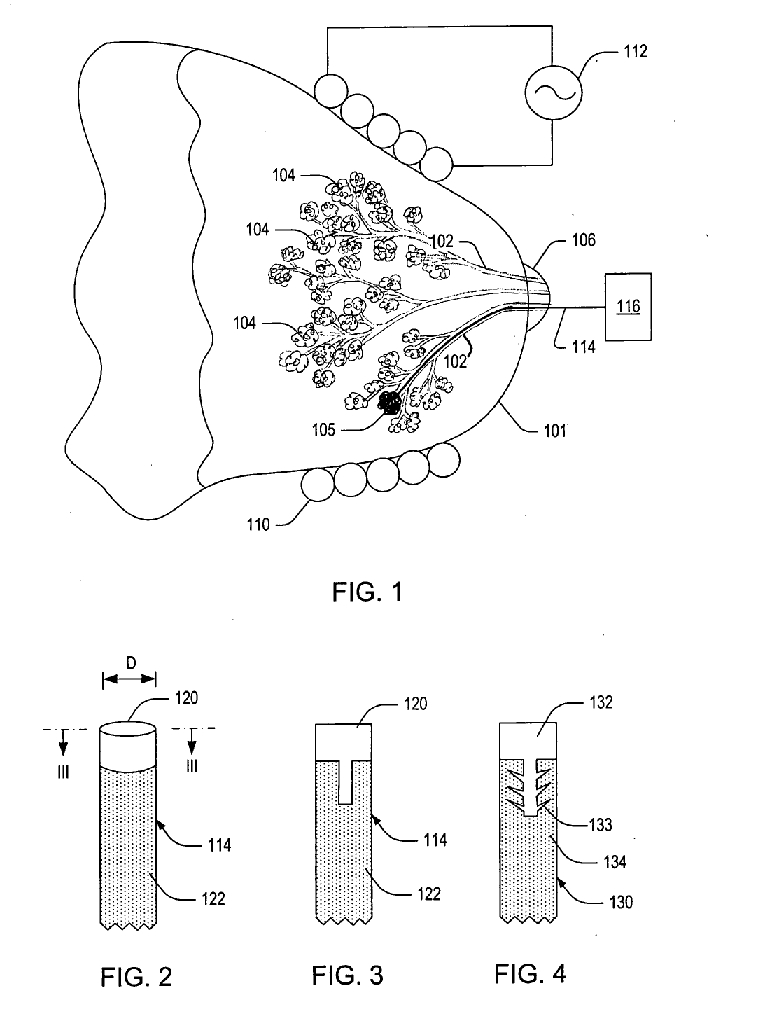

Referring now to FIG. 1, FIG. 1 shows a schematic diagram of a system for treating human breast cancer in accordance with one embodiment of the present invention. As depicted, a human breast 101 has a nipple 106; and ductal networks 102 that extend inwardly from the nipple and then into branching networks. Each network 102 includes a series of successively smaller lumens which are arranged in three dimensional configurations. Attached to the end of the smallest lumen is a lobule 104 for generating milk.

One type of breast cancer is Lobular carcinoma in situ (LCIS). LCIS 105 means that abnormal cells are contained within one or more of the lobules, but they haven't invaded the surrounding breast tissue. LCIS 105 is an early form of breast cancer or is just a marker for the future development of cancer. A patient having LCIS is at an increased risk of developing breast cancer in either breast in the future. In the breast 101 that has the LCIS 105, the patient is more likely to develop invasive lobular breast cancer. If cancer develops in the other breast, it's equally likely that it could be invasive lobular or invasive ductal carcinoma.

The treatment system in FIG. 1 includes a catheter 114 and an electrical coil 110 that generates an RF electromagnetic field when powered by an RF power source 112. A conventional circuit for controlling/operating the coil 110 can be used in the system, even though the conventional circuit is not shown in FIG. 1 for brevity. FIG. 2 shows a schematic perspective view of the catheter 114 including a tip portion 120 and a guide portion 122. The tip portion 120 of the catheter 114 is formed of material that inductively generates heat energy in response to the external RF field. As depicted in FIG. 1, the tip portion 120 of the catheter 114 is located in or nearby the LCIS 105 such that the heat energy generated by the tip 120 is used to necrose or ablate the LCIS 105. The intensity of the RF field, the coil operating frequency, the heat generator composition, and time interval for applying the RF field are determined by the type of cells and area to be treated by the system.

The catheter 114 has a generally elongated cylindrical shape. The diameter D of the tip portion 120 is determined by, inter alia, the dimension of intended applications, such as the size of the target tissue to be treated. The catheter 114 can be used to treat body tissue with higher precision than conventional catheter techniques. For instance, an existing technique includes filling the entire portion of a ductal network connected to a target lobule with fluid and heating the fluid to necrose the LCID as well as healthy lobules connected to the ductal network. In contrast, the treatment system in FIG. 1 allows the physician to treat the target LCIS 105 only. It is noted that the system in FIG. 1 can also treat other types of breast cancer, such as invasive lobular carcinoma (ILC). ILC starts in the milk-producing lobule 104 and invades the surrounding breast tissue. It can also spread to more distant parts of the body. By properly locating the tip portion 120 of the catheter 114 near the ILC and controlling the intensity of the electromagnetic field as well as the operational time of the coil 110, the invaded portion of the breast can be treated in a precise manner.

The tip portion 120 is inductively heated by the RF field of coil 110 to generate heat energy. In one exemplary embodiment, the tip portion 120 is formed of material that can generate heat energy in response to the electromagnetic field generated by the coil 110. The material for the tip portion 120 includes, but is not limited to, metal, plastic, polymer, ceramic, or alloys thereof. Some of the materials, such as metal, may have Curie temperature at which the material loses its magnetic properties. The type of material for the tip portion 120 is selected such that the Curie temperature of the material is at or below the predetermined operational temperature of the tip portion. Also, by properly selecting the material for the tip portion 120, the treatment system can selectively treat a specific type of abnormal cells while other types of cells remain intact during the treatment. For instance, the necrosis temperature of certain abnormal cells can be lower than that of healthy cells. In such a case, the Curie temperature of the material for the tip portion 120 is set between the two temperatures, allowing the system to discriminately necrose the abnormal cells.

The electrical coil 110 is formed of conventional conducting material. The coil 110 has a generally cylindrical shape and the diameter of the coil may vary along its center axis such that the inner surface of the coil can generally follow the outer profile of the breast 101. The operational frequency of the coil 110 is determined by the size and material of the tip portion 120 of the catheter 114 as well as the heat energy to be generated. For instance, the inductance (L) of the coil 110 is a function of, inter alia, the area enclosed by the coil and the resonance frequency of the LC tank circuit is determined by the values of L and capacitance (C). For a given C, the frequency of the tank circuit will decrease with a increasing value of L. From a functional point of view, the larger the coil diameter (for a constant current flow through the coil), the less homogenous the magnetic field within the coil. The magnetic field will be strongest near the coils and very weak in the center Thus, as will be discussed further in conjunction with FIG. 9, the coil 110 can have other geometrical shapes depending on the dimensions of target tissue and the organ surrounding the target tissue as well as the required strength of the RF field applied to the tip portion 120.

FIG. 3 shows a schematic cross sectional diagram of the catheter 114 in FIG. 2, taken along the line III-III. As depicted, the tip portion 120 is securely attached to the guiding portion 122 by suitable methods. For instance, adhesive material, such as cyanoacrylate and UV based adhesives, can be used to glue the tip portion 120 to the guide portion 122. Other methods include heat bonding (melting the guide portion material around the tip portion), mechanically crimping the tip portion to the guide portion, soldering a wire to the tip portion and having the wire run down the shaft of the catheter to a remote attachment point, and having features such as barbs or threads on the tip grip the ID (or OD) of the catheter.

Typically, the catheter 114 is inserted into a body until the tip portion 120 reaches the target tissue. Thus, the guiding portion 122 is formed of material that is flexible to bend and strong enough to support the tip portion during the insertion process or advancement of the tip portion toward the target tissue by the physician. The material for the guide portion 122 includes, but is not limited to, nylon and polyimide. In one exemplary embodiment, the guide portion 122 is formed of material that is transparent to the electromagnetic field generated by the coil 110.

FIG. 4 shows a schematic cross sectional diagram of another embodiment of a catheter 130. As depicted, the catheter 130 includes a tip portion 132 and a guide portion 134. To prevent unintentional disengagement of the tip portion 132 from the guide portion 134, the tip portion 132 has barbs 133 extending into the guide portion 134. The tip portion 132 and guide portion 134 may be formed of the same materials as the tip portion 120 and guide portion 122, respectively.

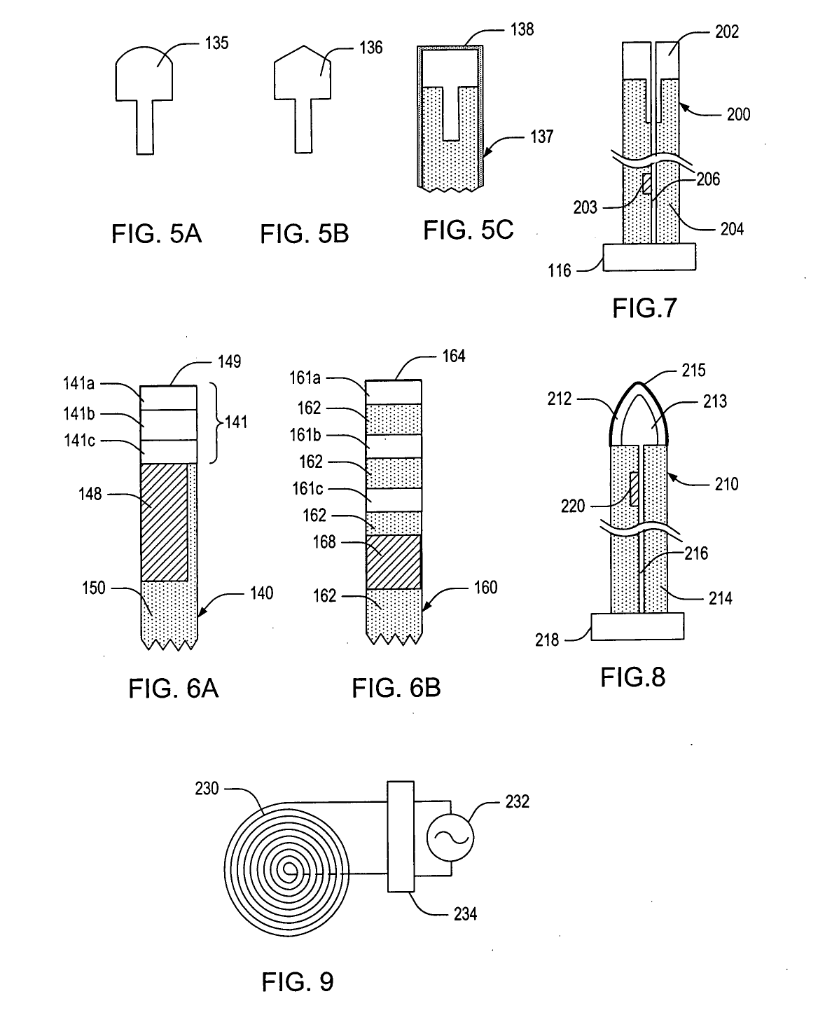

FIGS. 5A-5B show schematic cross sectional diagrams of various embodiments of the tip portion of the catheter 114. As depicted FIG. 5A, the distal end of the tip portion 135 is rounded to facilitate insertion of the catheter into an elongated lumen of a patient body, such as the ductal network 102 or blood vessel. In the case where the target tissue is localized in a small area, the tip portion 136 can have a sharp conical end as depicted FIG. 5B. It is noted that both of the tip portions 135 and 136 are glued to guide portions and/or have barbs that are similar to those 133 in FIG. 4.

FIG. 5C shows a schematic cross sectional diagram of another embodiment of a catheter. As depicted, the catheter 137 has a coating 138 to prevent direct contact between the tip/guide portions with the target tissue. The coating 138 also prevents tissue or coagulum from sticking to the heat generators, such as tip portion of the catheter, as well as improving the lubricity, heat transfer or abrasion resistance characteristics. The coating also prevents direct contact between the tip/guide portions with the patient body that the catheter 137 is inserted into, thereby reducing potential damages inflicted on the body. The coating 138 is, but not limited to, an anodization layer, a passivation layer, or polytetrafluoroethylene (PTFE) layer and transparent to the RF radiation generated by the coil 110.

FIG. 6A shows a schematic side view of another embodiment of a catheter 140. As depicted, the catheter 140 includes: a tip portion 141 that has three RF heat generators 141a-141c; and a guiding portion 150 with a coated surface portion 148. Hereinafter, the term RF heat generator (or, shortly, heat generator) collectively refers to a portion(s) of the catheter that generates heat energy in response to the external RF electromagnetic field generated by a coil. Also, RF heat generators are formed of material that is similar to that of the tip portion 120. Likewise, hereinafter, guide portions are formed of material that is similar to that of the guide portion 122. The dimensions and materials for the three RF heat generators 141a-141c are selected by the type of application. For instance, the RF heat generators may have different Curie temperatures such that the tip portion 141, when excited by a generally uniform electromagnetic field, can have a predesigned temperature distribution along the longitudinal axis of the catheter 140. It is noted that other suitable number of heat generators can be included in the catheter without deviating from the teachings of the present disclosure.

The shape and dimension of the coated surface portion 148 is also determined by the type of application, more specifically, the area of the tissue to be treated by the catheter 140. For instance, the coated surface portion 148 can be extended along the longitudinal axis of the catheter 140 to have a shape of generally circular cylindrical shell and is used to treat ductal carcimona in situ (DCIS). DCIS is a common type of breast cancer and refers to abnormal cells in the lining of a milk duct that haven't invaded the surrounding breast tissue. This is early-stage breast cancer and some experts consider DCIS a “precancerous” condition. If left untreated, DCIS may eventually develop into invasive breast cancer, i.e., the cancer cells may break free of where they originate and invade the surrounding tissues that support the ducts and lobules of the breast. The DCIS can be treated by disposing the coated surface portion of the catheter 140 within the lining of a milk duct having DCIS and applying external RF electromagnetic field to the coated surface portion.

The catheter 140 may have other suitable number and distribution of coated surface portions along the longitudinal axis thereof. As in the case of the tip portion 120, the coating may be formed of material that generates heat energy in response to the RF field formed by the coil 110 during operation.

In FIG. 6A, the exemplary catheter 140 is shown to have three RF heat generators 141a-141c and coated surface portion 148 in sequence, starting from the distal end 149 of the catheter. However, it is noted that the arrangement of the three RF heat generators 141a-1 141c and coated surface portion 148 along the longitudinal axis of the catheter can be changed depending on the type of application. FIG. 6B shows a schematic side view of yet another embodiment of a catheter 160. As depicted, the catheter 160 includes: three RF heat generators 161a-161c; a coated surface portion 168; and a guiding portion 162, wherein the distal end portion of the catheter 160 includes alternating segments of heat generators, coated surface portion, and guide portion. Each heat generator can be spaced apart from the distal end 164 of the catheter by a suitable length. The material for each element of the catheter 160 is similar to that of the corresponding element of the catheter 140. For instance, the coating of the coated surface portion 168 will be similar to the coating of the portion 148.

FIG. 7 shows a schematic cross sectional diagram of yet another embodiment of a catheter 200. As depicted, the catheter 200 includes a tip portion 202, a guide portion 204, a ductal lumen 206 extending from the distal end to the proximal end of the catheter 200 in the longitudinal direction of the catheter, and a port 116 coupled to the ductal lumen 206. Various types of fluid can be introduced and taken out through the port 116 via the ductal lumen 206. For instance, fluid for washing the ductal network 102 (FIG. 1) can be introduced through the port 116. For anther instance, fluid for distending the ductal network 102 can be injected through the port 116 at a preset pressure. For yet another instance, a dye or contrast substance, such as liquid containing Ba and/or Ni, for fluoroscopy can be injected through the port 116 so that the physician can precisely advance the catheter 200 to the target tissue to be treated. It is noted that the tip portion 202 of the catheter 200 may have other suitable number of RF heat generators and coated areas as depicted in FIGS. 6A-6B.

Optionally, the catheter 200 may include at least one RF heat generator 203 disposed within the guide portion 204. The heat generator 203 generates heat energy for heating the fluid within the ductal lumen 206 in response to an external RF field. It should be apparent to those of ordinary skill that the heat generator 203 can have any suitable shapes, such as, ring, elongated bar, hollow tube, or the like, and be formed of material similar to that of the tip portion 120. Alternatively, the heat generator 203 can be a coating applied to the inner surface of the guide portion and formed of material similar to the coating of the portion 148.

FIG. 8 shows a schematic cross sectional diagram of still another embodiment of a catheter 210. As depicted, the catheter 210 includes a tip portion 212, a guide portion 214, a ductal lumen 216, and a port 218 coupled to the ductal lumen 216. The tip portion 212 includes a balloon for angioplasty, such as, coronary angioplasty to open narrowed or clogged blood vessels of the heart. The tip portion 212 is formed of inflatable material and has a cavity 213 that is in fluid communication with the ductal lumen 216. Fluid for opening the vessels is introduced through the port 218 to the cavity 213, inflating the tip portion 212 during operation. The tip portion 212 is also coated with ferromagnetic material 215, such as metal, for generating heat energy in response to the external RF electromagnetic field generated by a coil. The guide portion 214 is formed of material that is transparent to the external RF electromagnetic field. The top portion 212 is formed of flexible material, such as Nylon 11, 12, 66, polycarbonate, polyethylene, polypropylene, polyurethane, vinyl, polyvinyl chloride, Acrylonitrile Butadiene Styrene, Pebax®, Hytrel®, C-Flex®, Texin®, and Tecoflex®, that can stand the Curie temperature of the coating applied thereto. It is noted that the catheter 210 may have additional coatings formed on the guide portion 214 and additional RF heat generators arranged along the longitudinal axis thereof.

Optionally, the catheter 210 may include at least one RF heat generator 220 disposed within the guide portion 214. The heat generator 220 generates heat energy for heating the fluid within the ductal lumen 216 in response to an external RF field. It should be apparent to those of ordinary skill that the heat generator 220 can have any suitable shapes, such as, ring, elongated bar, hollow tube, or the like, and be formed of material similar to that of the tip portion 120. Alternatively, the heat generator 220 can be a coating applied to the inner surface of the guide portion and formed of material similar to the coating of the portion 148.

In FIG. 1, the coil 110 is shown to have a generally circular cylindrical shape. However, if the tissue to be treated is near the body skin and thereby the heat generators of the catheter 114 are located near the body skin, a different type of coil may be used. FIG. 9 shows a schematic top view of an exemplary embodiment of an electrical coil 230 that may be used to excite the heat generators of a catheter located near the body skin. As depicted, the coil 230 has a generally circular planar shape and coupled to an RF power source 232 via a circuit 234 for controlling/operating the coil. During operation, the coil 230 is moved near the heat generators of a catheter so that the heat generators can inductively generate heat energy to treat the target tissue. For brevity, other configurations of the coil are not detailed in the present document. However, it should be apparent to those of ordinary skill that the coil may have other suitable configurations depending on the type of target tissue and the body configuration surrounding the target tissue.

It is noted that the catheters shown FIGS. 1-9 can be used to treat various types of target tissue. For instance, one of the catheters in FIGS. 1-9, such as 114 in FIG. 2, is inserted into blood vessels and the tip portion 120 is excited by the external RF electromagnetic field such that the heat energy generated by the tip portion 120 shrinks a portion of the blood vessel and thereby to close the blood vessel. For another instance, incontinence can be treated by use of a catheter, such as 140 in FIG. 6A. A patient with incontinence loses urine involuntarily during physical activities that put pressure on the abdomen. The target tissue/muscle that does not close properly, such as weakened sphincter, bladder neck, or urethra, can be heated by the catheter 140 to shrink to an intended size such that the target tissue can restore urinary control. For brevity, the other types of treatments are not detailed in the present disclosure. However, it should be apparent to those of ordinary skill that the catheters in FIGS. 1-9 can be applied to various types of treatments.

It should be understood, of course, that the foregoing relates to exemplary embodiments of the invention and that modifications may be made without departing from the spirit and scope of the invention as set forth in the following claims.

What is claimed is:

1. A catheter, comprising:
an elongated cylindrical guide portion having a distal end and a proximal end; and
one or more heat generators securely attached to said elongated guide portion and operative to inductively generate heat energy in response to an electromagnetic field externally applied thereto,
wherein at least one of said heat generators is disposed near tissue so that the heat energy is used to treat the tissue during operation.

2. A catheter as recited in claim 1, wherein said heat generators include a tip portion secured to said distal end.

3. A catheter as recited in claim 2, wherein said tip portion is secured to said guide portion by a plurality of barbs or glue.

4. A catheter as recited in claim 2, wherein a distal end of said tip portion is pointed or rounded.

5. A catheter as recited in claim 1, wherein each said heat generator has a Curie temperature.

6. A catheter as recited in claim 1, wherein a portion of said catheter is coated with material for preventing direct contact between the tissue and said catheter.

7. A catheter as recited in claim 6, wherein said material is transparent to the electromagnetic field.

8. A catheter as recited in claim 1, wherein said heat generators include one or more coated surface portions of said guide portion that are operative to generate heat energy in response to the electromagnetic field.

9. A catheter as recited in claim 1, further comprising:
a ductal lumen extending from said distal end to said proximal end along a logitudinal direction of said guide portion.

10. A catheter as recited in claim 9, further comprising:
a port coupled to said guide portion at said proximal end and in fluid communication with said ductal lumen.

11. A catheter as recited in claim 9, wherein said ductal lumen is configured to have fluid therein and wherein at least of one of said heat generators is operative to heat the fluid.

12. A catheter as recited in claim 1, further comprising:
a ductal lumen extending from said distal end to said proximal end along a logitudinal direction of said guide portion,
wherein said heat generators include a tip portion secured to the distal end, said tip portion having a cavity in fluid communication with said ductal lumen and being adapted to inflate when said cavity is subject to an internal pressure applied through said ductal lumen, and wherein said tip portion has a coating applied on the outer surface thereof and operative to generate the heat energy.

13. A catheter as recited in claim 1, wherein the electromagnetic field is generated by a coil coupled to an RF power source.

14. A catheter as recited in claim 1, wherein said heat generators are disposed in said guide portion and distributed along a longitudinal direction of said guide portion.

15. A catheter as recited in claim 1, wherein said heat generators are formed of material selected from the group consisting of metal, plastic, polymer, ceramic, and alloys thereof.

16. A catheter as recited in claim 1, wherein said guide portion is formed of a material that is transparent to the electromagnetic field.

17. A system for treating tissue, comprising:
a coil for generating an alternating electromagnetic field; and
a catheter including:
an elongated cylindrical guide portion; and
one or more heat generators securely attached to said elongated guide portion and operative to inductively generate heat energy in response to the electromagnetic field,
wherein at least one of said heat generators is disposed near tissue so that the heat energy is used to treat the tissue during operation.

18. A method for treating tissue, said method comprising:
positioning a heat generator of a catheter near tissue to be treated; and
applying an external electromagnetic field to the heat generator to cause said heat generator to inductively generate heat energy in response to the electromagnetic field thereby treating the tissue by the heat energy.

19. A method as recited in claim 18, wherein the tissue includes abnormal tissues and the step of treating the tissue includes necrosing the abnormal tissue.

20. A method as recited in claim 18, wherein the step of treating the tissue includes shrinking the tissue to an intended size.
